(19) United States
(12) Patent Application Publication
(10) Pub. No.: US 2005/0201949 A1
(43) Pub. Date: Sep. 15, 2005

(54) Pharmaceutical formulations

(76) Inventors: Michael John Monteith, Stevenage (GB); Anthony James Taylor, Ware (GB); Marian Thomas, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/023,015

(22) Filed: Dec. 21, 2004

Related U.S. Application Data
(63) Continuation of application No. PCT/EP04/07668, filed on Jul. 8, 2004.
(60) Provisional application No. 60/505,406, filed on Sep. 23, 2003.

(30) Foreign Application Priority Data
Jul. 11, 2003 (GB) 0316335.9

(51) Int. Cl. A61L 9/04; A61K 9/14; A61K 31/16; A61K 31/715

(52) U.S. Cl. 424/46; 514/54; 514/630

(57) ABSTRACT

The invention relates to a solid pharmaceutical formulation comprising (a) an active ingredient substance susceptible to chemical interaction with lactose, (b) a carrier and (c) a ternary agent that is a sugar ester is also provided together with uses thereof and methods related thereto. The use of a ternary agent that is a sugar ester to inhibit or reduce chemical interaction between said active ingredient substance and a carrier in a solid pharmaceutical formulation, wherein the active ingredient substance is susceptible to chemical interaction with the carrier and the ternary agent is a sugar ester.

The present application is a continuation-in-part application of PCT Application No. PCT/EP2004/007668 filed Jul. 8, 2004, which claims priority to GB Application No. 0316335.9 filed Jul. 11, 2003, the disclosures of which are incorporated herein by reference in their entirety.

The present invention relates to novel solid pharmaceutical formulations which comprise as active ingredient drug substance a long-acting beta-agonist as defined hereinbelow, a carrier and ternary agent which is a sugar ester. In particular the present invention relates to a novel solid pharmaceutical formulation comprising a compound (I) as defined hereinbelow, lactose and cellobiose octaacetate. The invention also relates to the use of said ternary agent for the stabilisation of said active ingredient drug substance in the presence of a carrier.

International Patent Application WO 01/42193 and corresponding U.S. Pat. No. 6,576,793 disclose inter alia a novel compound of the formula (I):

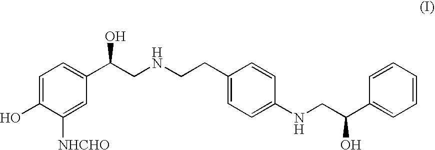

Compound (I) may variously be referred to by the chemical names N-{2-[4-((R)-2-hydroxy-2-phenylethylamino)phenyl]ethyl}-(R)-2-hydroxy-2-(3-formamido-4-hydroxyphenyl) ethylamine; N-[3-[(1R)-1-hydroxy-2-[[2-[4-[((2R)-2-hydroxy-2-phenylethyl)amino]phenyl]ethyl]amino]ethyl-6-hydroxyphenyl]-formamide and (α-R)-3-formamido-4-hydroxy-(α-[[[p-(N-((2R)-hydroxy-phenethyl))-amino-phenethyl]amino]methyl benzyl alcohol. In CAS format the compound (i) is designated: formamide, N-[2-hydroxy-5-[(1R)-1-hydroxy-2-[[2-4-[[(2R)-2-hydroxy-2-phenylethyl]amino]phenyl]ethyl]amino]ethyl]phenyl]-,.

Compound (I) is a potent and selective β₂ adrenergic receptor agonist, and as such may be useful in the prophylaxis and treatment of clinical conditions for which a selective β₂-adrenoreceptor agonist is indicated, including diseases associated with reversible airways obstruction such as asthma, chronic obstructive pulmonary diseases (COPD) (e.g. chronic and wheezy bronchitis, emphysema), respiratory tract infection and upper respiratory tract disease.

Other conditions which may be treated include premature labour, depression, congestive heart failure, skin diseases (e.g. inflammatory, allergic, psoriatic, and proliferative skin diseases), conditions where lowering peptic acidity is desirable (e.g. peptic and gastric ulceration) and muscle wasting disease.

WO01/42193 describes various pharmaceutical formulations, including those for inhalation, inter alia dry powder formulations.

An important requirement of pharmaceutical formulations is that they should be stable on storage in a range of different conditions. It is known that active ingredient substances can demonstrate instability to one or more of heat, light or moisture and various precautions must be taken in formulating and storing such substances to ensure that the pharmaceutical products remain in an acceptable condition for use over a reasonable period of time, such that they have an adequate shelf-life. Instability of a drug substance may also arise from contact with one or more other components present in a formulation, for example a component present as an excipient.

It is usual practice in the pharmaceutical art to formulate active ingredient substance with substances known as excipients which may be required as carriers, diluents, fillers, bulking agents, binders etc. Such excipients are often used to give bulk to a pharmaceutical formulation where the active ingredient substance is present in very small quantities. Such substances are generally chemically inert. Over prolonged storage times, or under conditions of extreme heat or humidity, and in the presence of other materials, such inert substances can, however, undergo or participate in chemical degradation reactions.

Carrier substances that are commonly utilised in solid pharmaceutical formulations include reducing sugars, for example lactose, maltose and glucose. Lactose is particularly commonly used. It is generally regarded as an inert excipient.

However, it has been observed that certain active ingredient substances may undergo a chemical reaction in the presence of lactose and other reducing sugars. For example, it was reported by Wirth et al. (J. Pharm. Sci., 1998, 87, 31-39) that fluoxetine hydrochloride (sold under the tradename Prozac®) undergoes degradation when present in solid tablets with a lactose excipient. The degradation was postulated to occur by formation of adducts via the Maillard reaction and a number of early Maillard reaction intermediates were identified. The authors conclude that drug substances which are secondary or primary amines undergo the Maillard reaction with lactose under pharmaceutically relevant conditions.

The present inventors have found that, under accelerated stability conditions, certain inhalable active ingredient substances also undergo degradation in the presence of lactose, possibly also via the Maillard reaction.

WO 96/23485 (Coordinated Drug Development Ltd), WO01/78694 and WO01/78695 (Vectura Limited) each describe a powder for use in a dry powder inhaler including an active ingredient particles and carrier particles, wherein the carrier includes an additive which is able to promote release of the active particles from the carrier particles. Possible additive materials include amino acids, phospholipids, and surface active agents including inter alia sugar esters.

We have now found that chemical interaction of active ingredient substance and carrier may be inhibited or reduced by the presence of a ternary agent which is a sugar ester as described below. In particular, we have found that dry powder formulations comprising compound (I), lactose and cellobiose octaacetate demonstrate reduced degradation and hence enhanced stability as compared to similar formulations without cellobiose octaacetate.

In a first aspect therefore the present invention provides a solid pharmaceutical formulation comprising (a) an active ingredient substance, (b) a carrier and (c) a ternary agent that is a sugar ester, wherein said active ingredient substance is the compound of formula (I): 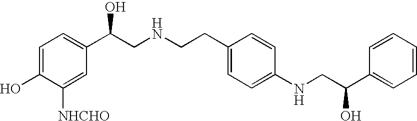
or a pharmaceutically acceptable salt, solvate or hydrate thereof.

In a particular embodiment the present invention provides a solid pharmaceutical formulation comprising (a) the compound of formula (I) or a pharmaceutically acceptable salt, solvate or hydrate thereof, (b) lactose and (c) cellobiose octaacetate.

In this specification references to the compound of formula (I) will extend to pharmaceutically acceptable salts, solvates and hydrates unless the context dictates otherwise.

In a second aspect the present invention provides the use of a ternary agent which is a sugar ester, e.g cellobiose octaacetate, to inhibit or reduce chemical interaction between the compound of formula (I) and a carrier, e.g. lactose, in a solid pharmaceutical formulation, wherein the active ingredient substance is susceptible to chemical interaction with the carrier.

The invention also provides in a third aspect the use of a ternary agent which is a sugar ester, e.g. cellobiose octaacetate, to inhibit or reduce chemical degradation of the compound (I) in a solid pharmaceutical formulation comprising the compound of formula (I) and a carrier, e.g. lactose, wherein the compound of formula (I) is susceptible to chemical interaction with said carrier.

In a fourth aspect the present invention provides a method of reducing or inhibiting chemical interaction between the compound of formula (I) and a carrier susceptible to chemical interaction, e.g. lactose, which comprises mixing with said compound (I) and said carrier a ternary agent that is a sugar ester, e.g. cellobiose octaacetate. The invention also provides a method of inhibiting chemical degradation of compound (I) in a formulation comprising a carrier, e.g. lactose and compound (I), which method comprises mixing with compound (I) and said carrier a ternary agent that is a sugar ester, e.g. cellobiose octaacetate.

An example of an ester of a sugar which may be employed in the present invention is cellobiose octaacetate.

Pharmaceutical formulations that have been prepared according to the present invention have greater chemical stability than the corresponding formulations without said sugar ester.

In the context of the present invention the sugar ester may be referred to as a ternary agent. ‘Ternary agent’ is used herein to mean a compound used in a formulation in addition to the active ingredient drug substance or substances (the ‘primary’ agent) and a bulk carrier material or materials (the ‘secondary’ agent). In some circumstances more than one ternary agent may be used. Optionally, further substances, possibly named ‘quaternary agents’, may also be present, for example as a lubricant. Any particular ternary or quaternary agent may have more than one effect.

The invention finds particular application in formulations in which the carrier is a reducing sugar, for example lactose, maltose or glucose (for example monohydrate glucose or anhydrate glucose). In a preferred embodiment, the carrier is lactose. Alternative carriers include maltodextrin.

The optimal amount of ternary agent present in a particular composition varies depending on the identity of the sugar ester ternary agent, the sizes of the particles and various other factors. In general, the sugar ester is preferably present in an amount of from 0.1 to 20% w/w based on the total weight of the composition. More preferably the sugar ester is present in an amount of from 0.2 to 10% w/w based on the total weight of the composition. When cellobiose octaacetate is used as the ternary agent, it is preferably present in an amount of from 2 to 15% w/w, for example from 4 to 10% w/w.

The compound (I) is typically present in an amount of from 0.01% to 50% w/w based on the total weight of the composition. Preferably, compound (I) is present in an amount of from 0.02% to 10% w/w, more preferably in an amount of from 0.03 to 5% w/w, for example from 0.05% to 1% w/w, for example 0.1% w/w.

The compound of formula (i) may be in the form of a free acid or base or may be present as a salt, a solvate, or other physiologically acceptable derivative. Salts and solvates which are suitable for use in medicine are those wherein the counterion or associated solvent is pharmaceutically acceptable.

Suitable salts for use in the invention include those formed with both organic and inorganic acids or bases. Pharmaceutically acceptable acid addition salts include those formed from hydrochloric, hydrobromic, sulphuric, citric, tartaric, phosphoric, lactic, pyruvic, acetic, trifluoroacetic, triphenylacetic, phenylacetic, substituted phenylacetic eg. methoxyphenylacetic, sulphamic, sulphanilic, succinic, oxalic, fumaric, maleic, malic, glutamic, aspartic, oxaloacetic, methanesulphonic, ethanesulphonic, arylsulphonic (for example p-toluenesulphonic, benzenesulphonic, naphthalenesulphonic or naphthalenedisulphonic), salicylic, glutaric, gluconic, tricarballylic, mandelic, cinnamic, substituted cinnamic (for example, methyl, methoxy, halo or phenyl substituted cinnamic, including 4-methyl and 4-methoxycinnamic acid and α-phenyl cinnamic acid (E or Z isomers or a mixture of the two)), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic) and isethionic acids. Pharmaceutically acceptable base salts include ammonium salts, alkali metal salts such as those of sodium and potassium, alkaline earth metal salts such as those of calcium and magnesium and salts with organic bases such as dicyclohexyl amine and N-methyl-D-glucamine.

A particular salt for use in the present invention is the hydrochloride, e.g. the monohydrochloride salt of compound (I).

A physiologically functional derivative of a drug substance may also be used in the invention. By the term “physiologically functional derivative” is meant a chemical derivative of a compound of having the same physiological function as the free compound, for example, by being convertible in the body thereto. According to the present invention, examples of physiologically functional derivatives include esters, for example compounds in which a hydroxyl group has been converted to a C₁₋₆-alkyl, aryl, aryl C₁₋₆ alkyl, or amino acid ester.

Formulations to which the present invention may be applied include those suitable for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredient into association with the carrier and the ternary agent as well as any other accessory ingredients. In general the formulations are prepared by uniformly and intimately bringing into association the active ingredient, carrier, e.g. lactose, ternary agent and any other accessory ingredients, and then, if necessary, shaping the product into the desired formulation.

Formulations of the present invention suitable for oral administration may be presented as discrete units such as capsules, cachets or tablets each containing a predetermined amount of the active ingredient; as a powder or granules. The active ingredient drug substance may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include sterile powders, granules and tablets intended for dissolution immediately prior to administration. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose an acacia.

The invention finds particular application in dry powder compositions and in particular in dry powder compositions for topical delivery to the lung by inhalation.

Dry powder compositions for topical delivery to the lung by inhalation may, for example, be presented in capsules and cartridges of for example gelatine, or blisters of for example laminated aluminium foil, for use in an inhaler or insufflator. Packaging of the formulation may be suitable for unit dose or multi-dose delivery. In the case of multi-dose delivery, the formulation can be pre-metered (eg as in Diskus, see GB 2242134 or Diskhaler, see GB 2178965, 2129691 and 2169265) or metered in use (eg as in Turbuhaler, see EP 69715 or EP0237507). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing an active compound. Preferably, the strip is sufficiently flexible to be wound into a roll.

Medicaments for administration by inhalation desirably have a controlled particle size. The optimum particle size for inhalation into the bronchial system is usually 1-10 μm, preferably 2-5 μm (mass mean diameter, MMD). Particles having a size above 20 μm are generally too large when inhaled to reach the small airways. To achieve these particle sizes the particles of the active ingredient substance as produced may be size reduced by conventional means eg by micronisation. The desired fraction may be separated out by air classification or sieving. Preferably, the particles will be crystalline. In general, the particle size of the carrier, for example lactose, will be much greater than the drug substance within the present invention. It may also be desirable for other agents other than the active drug substance to have a larger particle size than the active drug substance. When the carrier is lactose it will typically be present as milled lactose, for example with a mass mean diameter (MMD) of 60-90 μm and with not more than 15% having a particle diameter of less than 15 μm.

The sugar ester will typically have a particle size in the range 1 to 50 μm, and more particularly 1-20 μm (mass mean diameter). The particle size of the sugar ester, e.g cellobiose octaacetate, for use in the preparation of compositions in accordance with this invention may be reduced by conventional methods to give particles with a mass mean diameter (MMD) in the range 1 to 10 μm, for example 1 to 5 μm. The sugar ester is typically micronised but may also be prepared using controlled precipitation, supercritical fluid methodology and spray drying techniques familiar to those skilled in the art.

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

The compounds and pharmaceutical formulations according to the invention may be used in combination with or include one or more other therapeutic agents, for example a beta-agonist may be used in combination with one or more other therapeutic agents selected from anti-inflammatory agents (for example a corticosteroid, or an NSAID,) anticholinergic agents (particularly an M₁, M₂, M₁/M₂ or M₃ receptor antagonist), other β₂-adrenoreceptor agonists, antiinfective agents (e.g. antibiotics, antivirals), or antihistamines.

Suitable corticosteroids include methyl prednisolone, prednisolone, dexamethasone, fluticasone propionate, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11-hydroxy-16α-methyl-3-oxo-17α-propionyloxy-androsta-1,4-diene-1 7β-carbothioic acid S-(2-oxo-tetrahydro-furan-3S-yl)ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide, butixocort propionate, RPR-106541, and ST-126.

Suitable NSAIDs include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis, iNOS inhibitors, tryptase and elastase inhibitors, beta-2 integrin antagonists and adenosine receptor agonists or antagonists (e.g. adenosine 2a agonists), cytokine antagonists (e.g. chemokine antagonists) or inhibitors of cytokine synthesis.

Suitable anticholinergic agents are those compounds that act as antagonists at the muscarinic receptor, in particular those compounds which are antagonists of the M₁ and M₂ receptors. Exemplary compounds include the alkaloids of the belladonna plants as illustrated by the likes of atropine, scopolamine, homatropine, hyoscyamine; these compounds are normally administered as a salt, being tertiary amines.

Preferred anticholinergics include ipratropium (e.g. as the bromide), sold under the name Atrovent, oxitropium (e.g. as the bromide) and tiotropium (e.g. as the bromide) (CAS-139404-48-1).

Suitable antihistamines (also referred to as H₁-receptor antagonists) include any one or more of the numerous antagonists known which inhibit H₁-receptors, and are safe for human use. All are reversible, competitive inhibitors of the interaction of histamine with H₁-receptors. Examples of preferred anti-histamines include methapyrilene and loratadine.

The invention further provides the use of an inhalable solid pharmaceutical formulation according to the invention for the manufacture of a medicament for the treatment of diseases associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, including seasonal and allergic rhinitis). The invention also provides a method for treating asthma, chronic obstructive pulmonary diseases (COPD), chronic or wheezy bronchitis, emphysema, respiratory tract infection upper respiratory tract, or rhinitis, including seasonal and allergic rhinitis comprising administering to a patient in need thereof an inhalable solid pharmaceutical formulation according to the invention.

In a further aspect, the invention provides a method of preparing a solid pharmaceutical preparation comprising combining in one or more steps: (a) compound (I), (b) a carrier and (c) a sugar ester.

### EXAMPLES

Test compound: In the following examples Compound (I) was used as the monohydrochloride salt

Method

Preparation of Blends

Lactose monohydrate was obtained from Borculo Domo Ingredients as BP/USNF form. Before use, the Lactose Monohydrate was sieved through a coarse screen (mesh size 500 microns) to deaggregate the material. Compound (I) was micronised before use in an APTM microniser to give a MMD (mean mass diameter) of from 2 to 5 microns.

Cellobiose octaacetate was obtained from Boregaard and was micronised before use. The cellobiose octaacetate was combined with lactose monohydrate and blended using a high shear mixer (Bohle or a TRV series mixer) to provide a cellobiose octaacetate/drug premix, hereinafter referred to as blend A.

Final blend B was obtained by first pre-mixing an appropriate quantity of blend A with compound (I) and then blending that blend A/compound (I) premix with further blend A in a weight ratio appropriate to provide blend B containing the cellobiose octaacetate in the required quantity, as indicated in Table 1 and Table 2 below. The quantities of compound (I), cellobiose octaacetate and lactose in Tables 1 and 2 are the amount by weight of each component present as a percentage of the total composition.

Decomposition Conditions

The blends prepared as described above were subjected to accelerated decomposition conditions in a controlled atmosphere stability cabinet. In the tables below, the conditions to which the blends were subjected are given with reference to the temperature and the % relative humidity, for example 30/60 is 30° C. and 60% relative humidity (RH). Samples were analysed for decomposition products after the time periods indicated in the tables.

Analysis of Purity of Blends after Subjection to Decomposition Conditions

LC analysis was conducted on a ACE5-C18 column (250×4.6 mm ID), 5 micron, eluting with water/acetonitrile (98v/2v) containing 0.05% trifluoroacetic acid (solvent A) and water/acetonitrile (10v/90v) containing 0.05% trifluoroacetic (solvent B) using the following elution gradient:time 0=94% solvent A, 6% solvent B; 25 mins=70% solvent A, 30% solvent B; 35 mins=40% solvent A, 60% solvent B; 37-39 mins=100% solvent B; 39.1-43 mins=94% solvent A, 6% solvent B. Flow rate was 1 ml/min and the column temperature was 30° C. Detection was carried out by UV at 244 nm with a Spectra series UV150 detector or a Shimadzu SPD-10A VP detector. The area under the LC trace curve for the total impurities was compared with the total area under the curve, to give the % area/area figures given in Tables 1 and 2.

Results

TABLE 1

| | | | Total |
|---|---|---|---|
| | | | impurities |
| | | | (% |
| Blend details | Timepoint | Condition | area/area) |
| 0.08% compound (I) with | Initial | | 0.89 |
| micronised Cellobiose | 1 Month | 40° C./75% RH | 1.22 |
| Octaacetate (7%) and 5% | 3 Months | 40° C./75% RH | 1.85 |
| lactose fine | | | |
| 0.32% compound (I) with | Initial | | 0.92 |
| micronised Cellobiose | 1 Month | 40° C./75% RH | 1.15 |
| Octaacetate (7%) and 5% | 3 Months | 40° C./75% RH | 1.18 |
| lactose fine | | | |

Results

TABLE 2

| | | | Total |
|---|---|---|---|
| | | | im- |
| | | | purities |
| | | | (% area/ |
| Blend details | Timepoint | Condition | area) |
| 0.08% compound (I) with 2% | Initial | / | 1.54 |
| micronised Cellobiose | 1 Month | 40° C./75% RH | 2.35 |
| octaacetate and 5% lactose fines | 3 Months | 40° C./75% RH | 2.42 |
| 0.08% compound (I) with 6% | Initial | / | 1.25 |
| micronised Cellobiose | 1 Month | 40° C./75% RH | 1.40 |
| octaacetate and 5% lactose fines | 3 Months | 40° C./75% RH | 1.60 |
| 0.08% compound (I) with 10% | Initial | / | 1.44 |
| micronised Cellobiose | 1 Month | 40° C./75% RH | 1.51 |
| octaacetate and 5% lactose fines | 3 Months | 40° C./75% RH | 1.50 |
| 0.08% compound (I) with 7% | Initial | / | 1.12 |
| micronised Cellobiose | 1 Month | 40° C./75% RH | 1.82 |
| octaacetate and 5% lactose fines | 3 Months | 40° C./75% RH | 2.42 |

1. A solid pharmaceutical formulation comprising (a) an active ingredient substance, (b) a carrier and (c) a ternary agent that is a sugar ester, wherein said active ingredient substance is the compound of formula (I):. 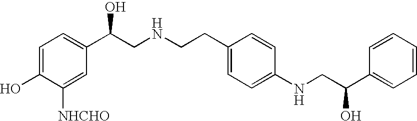
or a pharmaceutically acceptable salt, solvate or hydrate thereof.

2. A pharmaceutical formulation according to claim 1 wherein the carrier is lactose.

3. A pharmaceutical formulation according to claim 1 wherein the sugar ester is cellobiose octaacetate.

4. A pharmaceutical formulation according to claim 1 wherein the sugar ester is present in an amount of from 0.1 to 20% w/w based on the total weight of the composition

5. A pharmaceutical formulation according to claim 1 wherein the compound of the formula (i) is present in an amount of from 0.01% to 50% w/w based on the total weight of the composition.

6. An inhalable solid pharmaceutical formulation according to claim 1 further comprising one or more therapeutic agents in combination with the compound of formula (I).

7. A method of reducing or inhibiting chemical interaction between the compound of formula (I) as defined in claim 1 or a pharmaceutically acceptable salt, solvate or hydrate thereof and a carrier susceptible to chemical interaction, which comprises mixing a ternary agent which is a sugar ester with the compound of formula (I) and said carrier.

8. A method of reducing or inhibiting chemical degradation of the compound of formula (I) according to claim 1 or a pharmaceutically acceptable salt, solvate or hydrate thereof in a formulation comprising a carrier and the compound of formula (I), which method comprises mixing a ternary agent which is a sugar ester with the compound of formula (I) and said carrier.

9. A method as claimed in claim 7 wherein the sugar ester is cellobiose octaacetate.

10. A method as claimed in claim 8 wherein the sugar ester is cellobiose octaacetate.

11. A method for treating reversible airways obstruction comprising administering to a patient in need thereof a solid pharmaceutical formulation according to claim 1.

12. A method for treating asthma comprising administering to a patient in need thereof a solid pharmaceutical formulation according to claim 1.

13. A method for treating a chronic obstructive pulmonary disease comprising administering to a patient in need thereof a solid pharmaceutical formulation according to claim 1.

14. A method for treating reversible airways obstruction comprising administering to a patient in need thereof a solid pharmaceutical formulation according to claim 6.

15. A method for treating asthma comprising administering to a patient in need thereof a solid pharmaceutical formulation according to claim 6.

16. A method for treating a chronic obstructive pulmonary disease comprising administering to a patient in need thereof a solid pharmaceutical formulation according to claim 6.

17. A method of preparing a solid pharmaceutical preparation comprising combining (a) the compound of formula (I) according to claim 1 or a pharmaceutically acceptable salt, solvate or hydrate thereof, (b) a carrier and (c) a ternary agent that is a sugar ester.
